SEQUENCE LISTING (18 sequences)

SEQ ID NO: 1 - protein, 29 aa, synthetic construct
YCQKFLAPCDSXRKCCEGXECKLWCKKXX

SEQ ID NO: 2 - protein, 30 aa, synthetic construct
YCQKFLAPCDSXRKCCEGXECKLWCKKXXW

SEQ ID NO: 3 - protein, 30 aa, synthetic construct
YCQKFLAPCDSERKCCEGMECKLWCKKKLW

SEQ ID NO: 4 - protein, 30 aa, synthetic construct
YCQKFLAPCDSERKCCEGMECKLWCKKKIW

SEQ ID NO: 5 - protein, 29 aa, synthetic construct
YCQKFLAPCDSERKCCEGMECKLWCKKIW

SEQ ID NO: 6 - protein, 29 aa, synthetic construct
YCQKFLAPCDSERKCCEGMECKLWCKKII

SEQ ID NO: 7 - protein, 30 aa, synthetic construct
YCQKFLAPCDSERKCCEGLECKLWCKKKLW

SEQ ID NO: 8 - protein, 30 aa, synthetic construct
YCQKFLAPCDSERKCCEGLECKLWCKKKIW

SEQ ID NO: 9 - protein, 29 aa, synthetic construct
YCQKFLAPCDSERKCCEGLECKLWCKKIW

SEQ ID NO: 10 - protein, 29 aa, synthetic construct
YCQKFLAPCDSERKCCEGLECKLWCKKII

SEQ ID NO: 11 - protein, 30 aa, synthetic construct
YCQKFLAPCDSARKCCEGMECKLWCKKKLW

SEQ ID NO: 12 - protein, 30 aa, synthetic construct
YCQKFLAPCDSARKCCEGMECKLWCKKKIW

SEQ ID NO: 13 - protein, 29 aa, synthetic construct
YCQKFLAPCDSARKCCEGMECKLWCKKIW

SEQ ID NO: 14 - protein, 29 aa, synthetic construct
YCQKFLAPCDSARKCCEGMECKLWCKKII

SEQ ID NO: 15 - protein, 30 aa, synthetic construct
YCQKFLAPCDSARKCCEGLECKLWCKKKLW

SEQ ID NO: 16 - protein, 30 aa, synthetic construct
YCQKFLAPCDSARKCCEGLECKLWCKKKIW

SEQ ID NO: 17 - protein, 29 aa, synthetic construct
YCQKFLAPCDSARKCCEGLECKLWCKKIW

SEQ ID NO: 18 - protein, 29 aa, synthetic construct
YCQKFLAPCDSARKCCEGLECKLWCKKII

(19) United States
(12) Patent Application Publication
(10) Pub. No.: US 2026/0042802 A1
(43) Pub. Date: Feb. 12, 2026

(54) POLYPEPTIDE AND USE THEREOF

(71) Applicant: Guangzhou Xitao Biosciences Co., Ltd., Guangzhou (CN)

(72) Inventors: Haoyu Zeng, Guangzhou (CN); Sihua Hong, Guangzhou (CN); Jiexing Chen, Guangzhou (CN); Yun Li, Guangzhou (CN); Ting Fu, Guangzhou (CN); Tian Guan, Guangzhou (CN)

(21) Appl. No.: 19/363,697

(22) Filed: Oct. 21, 2025

Related U.S. Application Data
(63) Continuation of application No. PCT/CN2025/095128, filed on May 15, 2025.

(30) Foreign Application Priority Data
Mar. 26, 2024 (CN) 202410357014.2

(51) Int. Cl. C07K 14/00; A61K 38/00; A61P 25/04

(52) U.S. Cl. CPC C07K 14/00; A61P 25/04; A61K 38/00

(57) ABSTRACT

Provided in the present disclosure is a polypeptide and use thereof. The amino acid sequence of the polypeptide is as shown in SEQ ID NO: 1 or SE ID NO: 2; a 12^{th} amino acid in the amino acid sequence of the polypeptide is alanine or glutamic acid, a 19^{th} amino acid in the amino acid sequence of the polypeptide is methionine or leucine, a 28^{th} amino acid in the amino acid sequence of the polypeptide is lysine or isoleucine, and a 29^{th} amino acid in the amino acid sequence of the polypeptide is selected from any one of isoleucine, leucine and tryptophan. The polypeptide provided in the present disclosure provides good analgesic function and may selectively inhibit an activation of Naᵥ1.7 channels, thereby inhibiting pain, which is of great clinical importance in pain disorders.

### CROSS REFERENCE TO RELATED APPLICATIONS

The present application is a Continuation Application of PCT Application No. PCT/CN2025/095128 filed on May 15, 2025, which claims priority of Chinese Patent Applications No. 202410357014.2 filed on Mar. 26, 2024 before CNIPA. All the above are hereby incorporated by reference in their entirety as part of the present disclosure.

### REFERENCE TO SEQUENCE LISTING

This application includes a Sequence Listing filed electronically as an XML file named “TEST_SEQUENCE.xml”, created on Oct. 17, 2025, with a size of 17,011 bytes. The Sequence Listing is incorporated herein by reference.

### TECHNICAL FIELD

The present disclosure relates to the technical field of biomedicine and, specifically, to a polypeptide and use thereof.

### BACKGROUND

Voltage gated sodium channels (VGSCs) are multi-subunit transmembrane glycoproteins expressed in the cell membrane, consisting of α-subunit (functional unit) and β-subunit, α-subunit is composed of four homologous transmembrane domains (I˜IV), each of which contains six transmembrane hydrophobic α-helices (S1˜S6), and the positively-charged S4 fragment serves the voltage receptor function and is capable of regulating the hydrophilic channel in the middle of S5 and S6 that allows the passage of sodium ions, causing cellular depolarization or hyperpolarization and completing transmembrane transmission of signals.

In contrast to other sodium channel subtypes, Naᵥ1.7 channels offer the kinetics of rapid activation, rapid inactivation, and slow inactivation that may be activated by small stimuli to promote further depolarization of the cell membrane. The sodium channel expressed in peripheral neurons is predominantly Naᵥ1.7, and Naᵥ1.7 is more markedly expressed in injurious sensory neurons than in non-injurious sensory neurons.

Naᵥ1.7 is a sodium channel that was previously linked to the sensation of pain (i.e., nociception) through genetic studies in patients with rare pain disorders. Signals are sent from neurons to the brain and throughout the body by means of currents, and sodium channels are crucial to the cells' ability to generate these currents. When a neuron is stimulated, the Naᵥ1.7 channel opens, allowing positively-charged sodium ions to cross the cell membrane into a previously negatively-charged cell. The change in electric charge on the cell membrane produces a current that increases the excitability of the neuron and initiates a series of events that lead to pain. Therefore, the development of a polypeptide that selectively inhibits Naᵥ1.7 channel activation is of great clinical importance in pain disorders.

### SUMMARY

Provided in the present disclosure is a polypeptide and use thereof. The polypeptide provides good analgesic function and may selectively inhibit an activation of Naᵥ1.7 channels, thereby inhibiting pain, which is of great clinical importance in pain disorders.

In accordance with a first aspect of the present disclosure, provided is a polypeptide, in which: an amino acid sequence of the polypeptide is as shown in SEQ ID NO: 1 or SEQ ID NO: 2; a 12^{th} amino acid in the amino acid sequence of the polypeptide is alanine or glutamic acid; a 19^{th} amino acid in the amino acid sequence of the polypeptide is methionine or leucine; a 28^{th} amino acid in the amino acid sequence of the polypeptide is lysine or isoleucine; and a 29^{th} amino acid in the amino acid sequence of the polypeptide is selected from any one of isoleucine, leucine and tryptophan.

The polypeptide provided in the present disclosure provides good analgesic function and may selectively inhibit an activation of Naᵥ1.7 channels, thereby inhibiting pain, which is of great clinical importance in pain disorders.

Preferably, the amino acid sequence of the polypeptide mentioned above is selected from any one of SEQ ID NO: 3-18.

In accordance with a second aspect of the present disclosure, provided is a nucleic acid molecule, in which the nucleic acid molecule includes a nucleotide sequence encoding a polypeptide mentioned above.

In accordance with a third aspect of the present disclosure, provided is a recombinant expression vector, in which the recombinant expression vector includes the nucleic acid molecule mentioned above.

In accordance with a fourth aspect of the present disclosure, provided is a recombinant host cell, in which the recombinant host cell includes the recombinant expression vector mentioned above.

In accordance with a fifth aspect of the present disclosure, provided is an analgesic drug including a polypeptide mentioned above.

Preferably, the drug mentioned above is capable of inhibiting an activation of voltage gated sodium channels.

Preferably, the voltage gated sodium channels mentioned above include Naᵥ1.7 channels.

Preferably, a preparation type of the drugs with analgesic function mentioned above includes at least one of a colloidal solution type, an emulsion type, a suspension type, an oral instant film, an oral liquid, a capsule, an injection or a transdermal absorption preparation.

In accordance with a sixth aspect of the present disclosure, provided is a pharmaceutical composition, in which an active ingredient of the pharmaceutical composition includes a polypeptide mentioned above.

In accordance with a seventh aspect of the present disclosure, provided is an analgesic method, in which the method comprises the following steps: administering a polypeptide mentioned above to a subject.

The polypeptide provided in the present disclosure may selectively inhibit an activation of voltage gated Naᵥ1.7 sodium channels, thereby inhibiting pain. By applying the polypeptide provided in the present disclosure to the preparation of analgesic drugs, the produced drugs provide good analgesic effects and are of great clinical importance in pain disorders.

### BRIEF DESCRIPTION OF THE DRAWINGS

FIG. 1 is a graph showing the resulting effect of the polypeptide provided by the present disclosure on the current traces of Naᵥ1.7 overexpressing HEK293 cells.

FIG. 2 is a graph showing the resulting effect of the polypeptide PPN-01 on the current traces of Naᵥ1.4, Naᵥ1.5, Naᵥ1.6, and Naᵥ1.7 overexpressing HEK293 cells.

FIG. 3 is a graph of Naᵥ1.7 current traces at different concentrations of polypeptide PPN-01.

FIG. 4 is a graph of the inhibition results on the peak current of Naᵥ1.7 at different concentrations of polypeptide PPN-01.

FIG. 5 is a graph of the inhibition results of the polypeptide PPN-01 on the current-voltage dependence of Naᵥ1.7 channels.

FIG. 6 is a graph showing the resulting effect of the polypeptide provided by the present disclosure on the occurrence time of the first writhing and the number of writhing in mice.

FIG. 7 is a graph showing the resulting effect of polypeptide PPN-01 at different concentrations provided by the present disclosure on the number of writhing in mice.

FIG. 8 is a graph showing the resulting effects of the polypeptide PPN-01 at different concentrations provided by the present disclosure on the electrical signals of the sciatic nerve in rats.

### DETAILED DESCRIPTION

The technical features of the technical solutions in the present disclosure are clearly and completely described below in conjunction with the specific implementations. Obviously, the examples described herein are only some of the examples of the present disclosure but not all of them. Based on the examples in the present disclosure, all other examples obtained by those skilled in the art without creative efforts fall within the scope of protection of the present disclosure.

### Example 1

Provided in the present example is a polypeptide, whose amino acid sequence is as shown in SEQ ID NO: 1, in which a 12^{th} amino acid in the amino acid sequence of the polypeptide is alanine or glutamic acid, a 19^{th} amino acid in the amino acid sequence of the polypeptide is methionine or leucine, a 28^{th} amino acid in the amino acid sequence of the polypeptide is lysine or isoleucine, and a 29^{th} amino acid in the amino acid sequence of the polypeptide is selected from any one of isoleucine, leucine and tryptophan.

The amino acid sequences, as shown in SEQ ID NO: 1 and SEQ ID NO: 2, indicate the general formula of the polypeptide provided in the present disclosure, and amino acids at positions 12, 19, 28, and 29 are indicated by X in SEQ ID NO: 1 and SEQ ID NO: 2.

The difference between SEQ ID NO: 1 and SEQ ID NO: 2 is that SEQ ID NO: 1 contains a total of 29 amino acids, whereas SEQ ID NO: 2 adds an additional amino acid (W, i.e., tryptophan) to the N-terminus of the amino acid sequence of SEQ ID NO: 1.

TABLE 1
Amino acid sequence of the polypeptide

| | | | Amino acid sequence of the |
|---|---|---|---|
| | | | polypeptide (Ac→amid, i.e., |
| | | | from carboxyl terminus/ |
| | Title of the | | C-terminus→to amino |
| Groups | polypeptide | Sequence ID | terminus/N-terminus) |
| 1 | — | SEQ ID NO: 1 | YCQKFLAPCDSXRKCCEGXECKLWCKKXX |
| 2 | — | SEQ ID NO: 2 | YCQKFLAPCDSXRKCCEGXECKLWCKKXXW |
| 3 | PPN-01 | SEQ ID NO: 3 | YCQKFLAPCDSERKCCEGMECKLWCKKKLW |
| 4 | PPN-02 | SEQ ID NO: 4 | YCQKFLAPCDSERKCCEGMECKLWCKKKIW |
| 5 | PPN-03 | SEQ ID NO: 5 | YCQKFLAPCDSERKCCEGMECKLWCKKIW |
| 6 | PPN-04 | SEQ ID NO: 6 | YCQKFLAPCDSERKCCEGMECKLWCKKII |
| 7 | PPN-05 | SEQ ID NO: 7 | YCQKFLAPCDSERKCCEGLECKLWCKKKLW |
| 8 | PPN-06 | SEQ ID NO: 8 | YCQKFLAPCDSERKCCEGLECKLWCKKKIW |
| 9 | PPN-07 | SEQ ID NO: 9 | YCQKFLAPCDSERKCCEGLECKLWCKKIW |
| 10 | PPN-08 | SEQ ID NO: 10 | YCQKFLAPCDSERKCCEGLECKLWCKKII |
| 11 | PPN-09 | SEQ ID NO: 11 | YCQKFLAPCDSARKCCEGMECKLWCKKKLW |
| 12 | PPN-10 | SEQ ID NO: 12 | YCQKFLAPCDSARKCCEGMECKLWCKKKIW |
| 13 | PPN-11 | SEQ ID NO: 13 | YCQKFLAPCDSARKCCEGMECKLWCKKIW |
| 14 | PPN-12 | SEQ ID NO: 14 | YCQKFLAPCDSARKCCEGMECKLWCKKII |
| 15 | PPN-13 | SEQ ID NO: 15 | YCQKFLAPCDSARKCCEGLECKLWCKKKLW |
| 16 | PPN-14 | SEQ ID NO: 16 | YCQKFLAPCDSARKCCEGLECKLWCKKKIW |
| 17 | PPN-15 | SEQ ID NO: 17 | YCQKFLAPCDSARKCCEGLECKLWCKKIW |
| 18 | PPN-16 | SEQ ID NO: 18 | YCQKFLAPCDSARKCCEGLECKLWCKKII |

The present example lists in Table 1 several polypeptides and their corresponding amino acid sequences that meet the above requirements, and the polypeptides involved in the present disclosure are all prepared by the following steps:

1. 4-methylbenzhydrylamine (MBHA Resin, 1.0 mmol/g) is used to condense the amino acid links sequentially in accordance with the sequence from the C-terminus to the N-terminus of the polypeptide under the condition of S=0.3 mmol/g using the Fmoc synthesis process until the linear polypeptide condensation is completed to obtain a resin polypeptide. Then, acetic anhydride and pyridine are added to acetylate the N-terminus of the polypeptide, and the polypeptide is cut from the resin with a cutting solution (calculated in terms of volume ratio, trifluoroacetic acid (TFA): thioanisole: phenol: 1,2-Ethanedithiol (EDT): water=87.5:5:2.5:2.5:2.5) to obtain a linear polypeptide.

2. The linear polypeptide is added to acetonitrile solution (calculated in terms of volume ratio, acetonitrile: water=1:1) and mixed well to obtain a polypeptide solution with a concentration of 5 mg/ml. 60 mg of oxidized glutathione, 30 mg of reduced glutathione and 2.58 g of 4-hydroxyethylpiperazine ethane sulfonic acid (HEPES) are mixed well. The pH of the mixed system is further adjusted to 7.5 with 5M NaOH to obtain a folding solution.

3. 30 ml of the polypeptide solution mentioned above is taken, to which 90 ml of the folding solution is added, mixed homogeneously and placed in a refrigerator to react at 4° C. for 48 h. Samples are sampled and detected using high performance liquid chromatography, and purification is performed with the preparative liquid phase in accordance with the conditions shown in Table 2 after the reaction is basically completed.

TABLE 2
Preparation of liquid phase purification conditions

| Time (min) | A (0.1% TFA-acetonitrile) | B (0.1% TFA-water) |
|---|---|---|
| 0 | 10% | 90% |
| 5 | 20% | 80% |
| 45 | 40% | 60% |
| 50 | 80% | 20% |
| 56 | 10% | 90% |
| 60 | 10% | 90% |

### Example 2 Inhibition Effects of Different Polypeptides on Naᵥ1.7 Channels

The objective of the present example is to study the inhibition of Naᵥ1.7 channels by different polypeptides using patch clamp techniques.

The recording electrode used in the present example is a glass microelectrode, which is prepared as follows: The glass electrode blanks (outer diameter of 1.50 mm, inner diameter of 0.84 mm) are prepared into single-tube glass microelectrodes with tip diameters of 1-2 μm in four steps using the P-1000 Microelectrode Horizontal Puller for subsequent experiments. Be careful not to touch the heated platinum sheet in the center when pulling the electrode, and be careful not to touch the tip of the pulled electrode to prevent the tip from breaking. Since the tip of the glass microelectrode is highly susceptible to dust attraction, it is generally prepared for use now and should not be left overnight.

Glass microelectrodes are used as follows: rinse clean the glass microelectrodes before use; then put all the glass microelectrodes in a large beaker; add anhydrous ethanol so that the glass microelectrodes are completely immersed in anhydrous ethanol; and place the beaker in an ultrasonic cleaner with ultrasonic vibration for 30 min. Subsequently, pour out the anhydrous ethanol; rinse out the glass microelectrode with ultrapure water 3-4 times; use another small beaker to cover the microelectrode so as not to contaminate the electrode with dust; put the glass microelectrode into the oven for drying; and put the glass microelectrode into a clean box for spare use after drying.

In the patch clamp experiments, the measured sodium currents are corrected for leakage currents, and the primary data of the experiments are recorded using Clampfit 10.6, and the data acquisition is performed by the pCLAMP software. Three to five sweeps are selected for analysis, (sweep, which is a term used in patch clamp software and consists of a series of sampling points) in which the current is in a stable state before the addition of the polypeptide. The average value of the peak current is thus calculated as the control current amplitude. Then 3-5 sweeps in which the current is in a stable state after the addition of polypeptide are selected and analyzed, and the average value of the residual peak current is calculated as the residual current amplitude.

The electrical stimulation effect of polypeptides PPN-01 to PPN-16 on Naᵥ1.7 overexpressing HEK293 cells is studied using the glass microelectrodes obtained from the above preparations and the whole-cell patch clamp techniques (n=1). In a voltage-clamp mode, the clamp voltage is kept at −90 mV; a square-wave stimulus depolarized from −90 mV to +40 mV with a duration of 30 ms is applied; the concentration of each dosing (polypeptide) is 300 nM; and validation is repeated for the six polypeptides that show a significant effect (n=2).

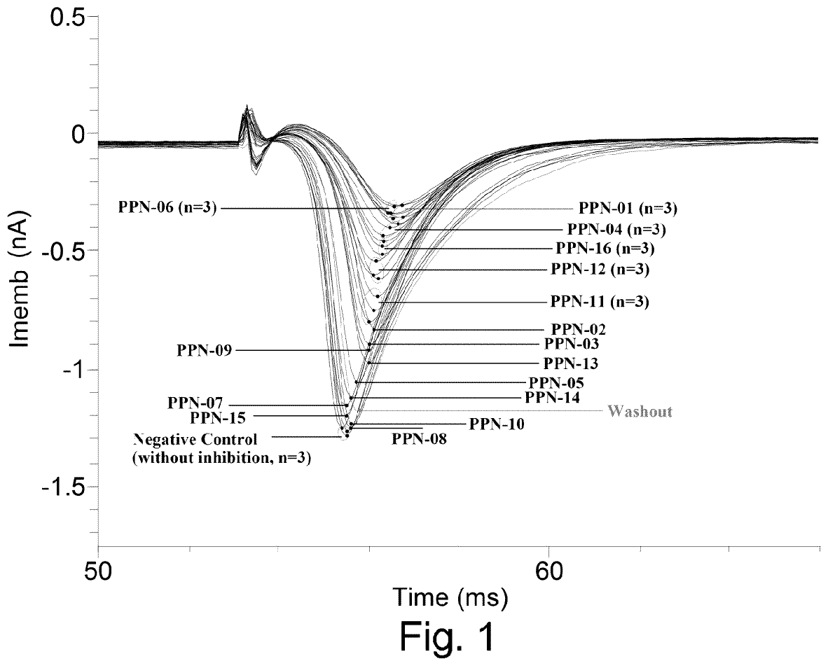

In a voltage-clamp mode, the clamp voltage is kept at −90 m V and a square-wave stimulus depolarized from −90 mV to +40 mV with a duration of 30 ms is applied. The results of the experiment are shown in FIG. 1. Compared to the negative control (no polypeptide added), the 16 polypeptides provided in Example 1 (PPN-01, PPN-02, PPN-03, PPN-04, PPN-05, PPN-06, PPN-07, PPN-08, PPN-09, PPN-10, PPN-11, PPN-12, PPN-13, PPN-14, PPN-15, and PPN-16) all indicate inhibition of Naᵥ1.7 overexpressing HEK293 cells, among which all six polypeptides, PPN-01, PPN-04, PPN-06, PPN-11, PPN-12, and PPN-16, indicate significant inhibition of Naᵥ1.7 overexpressing HEK293 cells.

### Example 3 Selectivity of Polypeptide PPN-01

The objective of the present example is to study the selectivity of the polypeptide PPN-01 provided in Example 1 for the voltage gated sodium channels Naᵥ1.4, Naᵥ1.5, Naᵥ1.6, and Naᵥ1.7.

The effect of the polypeptide PPN-01 provided in Example 1 on voltage gated sodium channels is studied using the glass microelectrodes prepared as described above in Example 2 and employing the whole-cell membrane clamp techniques. The specific operations are as follows:

1. Firstly, internal electrode solution is placed on ice to defrost; after defrosting, an appropriate amount of internal electrode solution is drawn up with a 1 ml syringe; and after a homemade filler is applied to the tip end of the syringe, the air in the tube is emptied to fill the entire filler with internal electrode solution. Secondly, internal electrode solution in the filler is poured into the glass microelectrode from a rear part thereof by using the rear part perfusion method; internal electrode solution is slowly injected into the glass microelectrode; and the amount of perfusion is generally ⅓ to ½ of a length of the glass microelectrode (it is necessary to ensure that the silver wire of the electrode may fully contact internal electrode solution, but also to avoid electrode contamination resulting from too much internal electrode solution). Thirdly, after the glass microelectrode is filled with internal electrode solution, a wall of the glass microelectrode tube should be lightly flicked in order to expel air bubbles, so that the tip of the glass microelectrode is filled with internal electrode solution. A resistance of the glass microelectrode after charging and filling is 4.0-6.0MΩ. Be careful not to use too much strength to flick the air bubbles and support the wall of the tube with the middle finger of the left hand, or else it is easy to break the glass microelectrode.

2. The electrical stimulation effect of the polypeptide PPN-01 provided in Example 1 on Naᵥ1.4, Naᵥ1.5, Naᵥ1.6, and Naᵥ1.7 overexpressing HEK293 cells is studied using the glass microelectrodes obtained from the above preparations and employing the whole-cell patch clamp techniques (n=5). In a voltage-clamp mode, the clamp voltage is kept at −90 mV; a square-wave stimulus depolarized from −90 mV to +40 mV with a duration of 30 ms is applied; and a concentration of each dosing (polypeptide) is 300 nM.

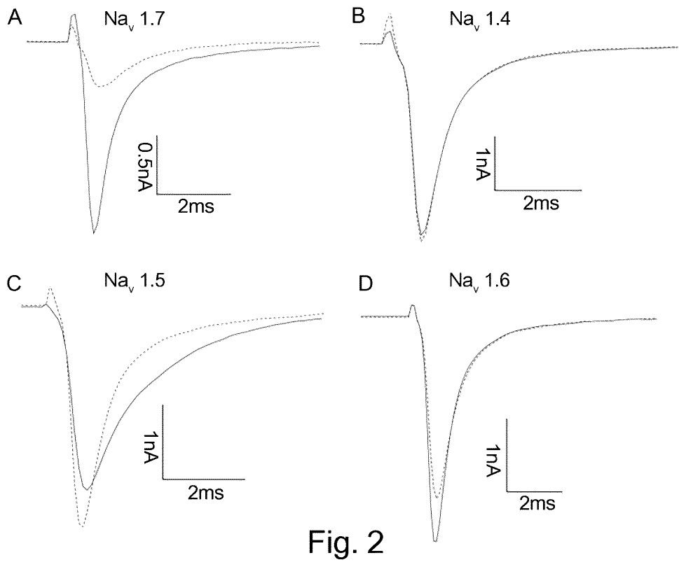

3. After establishing whole-cell patch clamp configuration using glass microelectrodes filled with internal electrode solution and HEK293 cells overexpressing Naᵥ1.4, Naᵥ1.5, Naᵥ1.6, or Naᵥ1.7, the polypeptides provided in Example 1 are applied separately. In a voltage-clamp mode, the clamp voltage is kept at −90 m V and a square-wave stimulus depolarized from −90 m V to +40 m V with a duration of 30 ms is applied. The experimental results show that the polypeptide PPN-01 provided in Example 1 provides a significant inhibition effect on Naᵥ1.7 overexpressing HEK293 cells, while it provides no significant inhibition effect on Naᵥ1.4, Naᵥ1.5, and Naᵥ1.6 overexpressing HEK293 cells. The current traces after the addition of the polypeptide PPN-01 with the amino acid sequence provided in Example 1 as shown in SEQ ID NO: 3 is shown in FIG. 2, in which the Naᵥ1.4, Naᵥ1.5, Naᵥ1.6, and Naᵥ1.7 overexpressing HEK293 cells are applied with a square-wave stimulus depolarized from −90 mV to +40 mV with a duration of 30 ms in a voltage-clamp mode.

As shown in FIG. 2, Naᵥ1.4, Naᵥ1.5, Naᵥ1.6, and Naᵥ1.7 overexpressing HEK293 cells (labeled as Naᵥ1.4, Naᵥ1.5, Naᵥ1.6, and Naᵥ1.7 groups respectively) are added to glass microelectrodes filled with internal electrode solution, and to which the polypeptide PPN-01 provided in Example 1 with the amino acid sequence shown in SEQ ID NO: 3 is added respectively. In a voltage-clamp mode, the clamp voltage is kept at −90 mV and a square-wave stimulus depolarized from −90 mV to +40 mV with a duration of 30 ms is applied. The current amplitude of the Naᵥ1.7 group (dotted line) shows significant reduced, while the current traces of the Naᵥ1.4, Naᵥ1.5, and Naᵥ1.6 groups (dotted lines) do not change significantly.

The above results indicate that the polypeptide PPN-01 provided in Example 1 is capable of selectively inhibiting activation of the voltage gated sodium channel Naᵥ1.7.

### Example 4 Inhibition by Different Concentrations of Polypeptide PPN-01 on Naᵥ1.7 Channels

The objective of the present example is to study the inhibition effect of different concentrations of polypeptide (the amino acid sequence of which is shown in SEQ ID NO: 3, hereinafter referred to as “polypeptide PPN-01” for convenience of description) on the Naᵥ1.7 channel using the patch clamp techniques.

In the patch clamp experiments (referring to Example 2), the measured sodium currents are corrected for leakage currents, and the primary data of the experiments are recorded using Clampfit 10.6, and the data acquisition is performed by the pCLAMP software. Three to five sweeps (sweep, which is a term used in patch clamp software and consists of a series of sampling points) in which the current is in a stable state before the addition of the polypeptide PPN-01 are selected for analysis. The average value of the peak current is thus calculated as the control current amplitude. Then 3-5 sweeps in which the current is in a stable state after the addition of polypeptide PPN-01 are selected and analyzed, and the average value of the residual peak current is calculated as the residual current amplitude. The inhibition rate of Naᵥ1.7 currents by polypeptide PPN-01 is calculated in accordance with the following equation: inhibition rate (%)=[1−(residual current amplitude)/(control current amplitude)]×100%.

The concentration-response curves are fitted by the Hill equation: I_{drug}/I_{control}=1/[1+ (C/IC₅₀)_{H}],

in which I_{control} is the average current amplitude in the control group, I_{drug} is the average current amplitude at different drug concentrations C (i.e., different concentrations of polypeptide-17), IC₅₀ is the drug concentration (i.e., polypeptide concentration) required to block 50% of the Naᵥ1.7 channels, and His the Hill coefficient.

The inward sodium current curves are obtained by fitting the rising and decaying segments using either a mono-exponential or bi-exponential equation for data from 90% to 10% between peak and baseline:

$$I ( t ) = {\sum }_{i = 1}^{n} {A}_{i} {e}^{- t / {τ}_{i}} + C ,$$

in which Aᵢ and τᵢ are an initial current amplitude and an activation/inactivation time constant respectively, and C is a non-time-dependent component.

Both activation and inactivation curves for Naᵥ1.7 channels are fitted with the Boltzmann equation: G=Gₘₐₓ/[1+exp ((V_{1/2}−Vₘ)/k)],

in which G is a sodium conductance, Gₘₐₓ is a maximum value of the sodium conductance, V_{1/2} is a voltage at which half of the channel is activated, Vₘ is a clamp voltage, and k is a slope factor.

The sodium conductance G is determined in accordance with the following equation: G=I/(Vₘ−Vᵣₑᵥ),

in which G is the sodium conductance, I is the current, Vₘ is the clamp voltage, and Vᵣₑᵥ is the reversal potential of the sodium current and is calculated in accordance with the Nernst equation to be +66 m V.

All statistics in the present disclosure are performed using one-way ANOVA for statistics between two groups, with P<0.05 as statistically different (* indicates P<0.05, ** indicates P<0.01, *** indicates P<0.001). All statistics are graphed using Graphpad Prism 9.0 software.

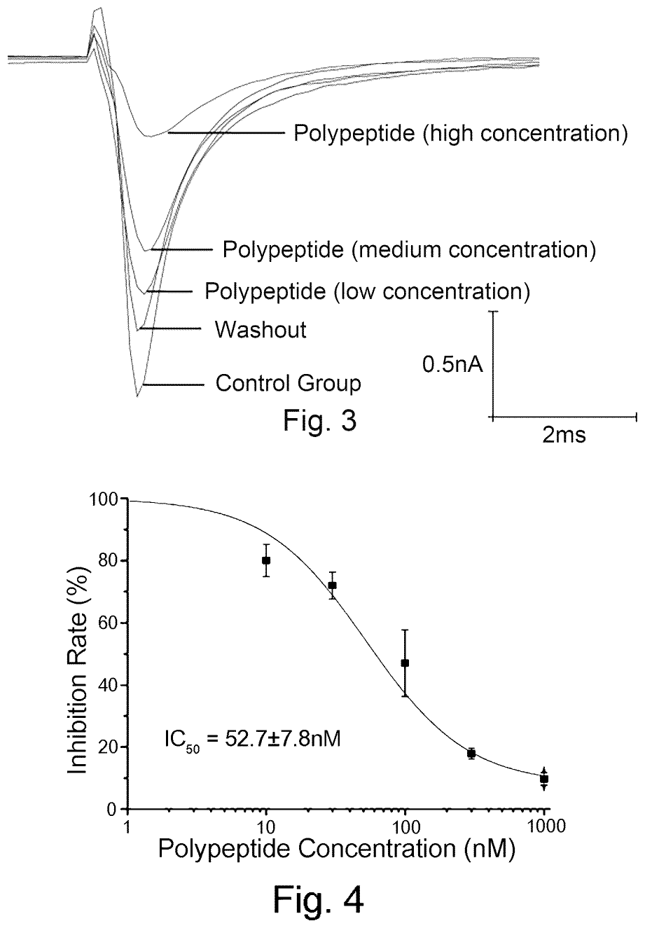

Representative Naᵥ1.7 current trace graphs (n=7) of currents led by a 30 ms square-wave stimulus from a clamp voltage of −90 m V depolarized to +40 mV, recorded before and after dosing of the polypeptide PPN-01 at different concentrations (low: 30 nM; medium: 100 nM; high: 300 nM), are shown in FIG. 3, in which the control group indicates that no dosing is performed.

Corresponding concentration-response curves are plotted in accordance with the inhibition percentage of Naᵥ1.7 peak current by polypeptides at each concentration, while the data are fitted in accordance with the steps described above, and the results are shown in FIG. 4.

As shown in FIGS. 3 and 4, the IC50 value of the polypeptide PPN-01 for inhibiting the peak current of Naᵥ1.7 is 52.7±7.8 nM, and the Hill coefficient is 1.18. The above results indicate that there is no intermolecular synergism in the interaction between the polypeptide PPN-01 and the Naᵥ1.7 channel.

### Example 5 Voltage Dependence of Naᵥ1.7 Channel Blockade by Polypeptide PPN-01

The objective of the present example is to study the voltage dependence of Naᵥ1.7 channel blockade by the polypeptide PPN-01 (the amino acid sequence of which is shown in SEQ ID NO: 3) using the patch clamp techniques.

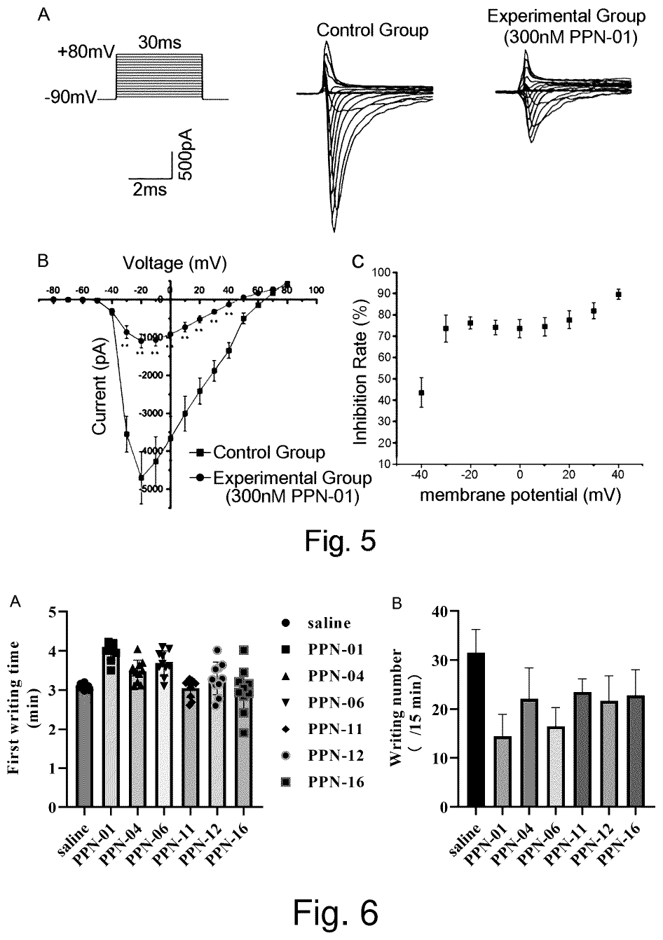

In a voltage-clamp mode, the clamp voltage is kept at −90 m V, and a square-wave stimulus depolarized from −90 mV to +40 mV with a step voltage of 10 mV and with a duration of 30 ms is applied to the Naᵥ1.7 overexpressing HEK293 cells to lead to the current traces at different voltage. The addition of 300 nM polypeptide PPN-01 is taken as the experimental group, and without the addition of 300 nM polypeptide PPN-01 is taken as the control group. The I-V relationship curve related to Naᵥ1.7 current (n=6, P<0.05) is constructed by graphing the peak amplitude of Naᵥ1.7 current versus the membrane potential, and also graphing the inhibition percentage versus the membrane potential, with the results shown in FIG. 5. Panel A of FIG. 5 represents the Naᵥ1.7 current traces led by a square-wave series stimulus of 30 ms duration depolarized from a clamp voltage of −90 mV to +80 mV (with a step voltage of 10 mV). Panel B of FIG. 5 represents the I-V (current-voltage) relationship curves of Naᵥ1.7 channels before and after polypeptide PPN-01 dosing. Panel C of FIG. 5 represents a graph of the current inhibition percentage led by the 300 nM polypeptide PPN-01 versus membrane potential.

As shown in inset A of FIG. 5, current inhibition is observed at all membrane potentials that activate the Naᵥ1.7 channel, suggesting that the current block induced by the polypeptide PPN-01 occurs throughout the range of potential activation. As shown in inset B and inset C of FIG. 5, the polypeptide PPN-01 may produce voltage-dependent inhibition between currents −40 mV and +40 mV, with current inhibition increasing from 43.57%±6.95% at −40 mV to 73.57%=6.26% at −30 mV (n=6, P<0.01), and then increasing from 73.57%±6.26% at −30 mV to 89.59%±2.46% at +40 mV (n=6, P<0.05). The above results suggest that the polypeptide PPN-01 provides a stronger inhibition of Naᵥ1.7 channels that are in an open state.

### Example 6 Effects of Different Polypeptides on the Mouse Acetate Acid Writhing Model

The objective of the present example is to study the effects of different polypeptides on the mouse acetate acid writhing model.

Seventy SPF-grade healthy C57BL/6 mice, half male and half female, weighing 22-25 g and 8-10 weeks old, are randomly selected. Seventy C57BL/6 mice are weighed and numbered in ascending order of body weight (No. S1-S70). The mice are randomly divided into seven groups in terms of the randomized numerical table method, which are the saline group and the experimental groups, among which the experimental groups include the PPN-01 group, the PPN-04 group, the PPN-06 group, the PPN-11 group, the PPN-12 group, and the PPN-16 group. The dosing concentration (i.e., the concentration of added polypeptides PPN-01, PPN-04, PPN-06, PPN-11, PPN-12, and PPN-16) in each group is 10 mg/kg, with 10 mice in each group.

Saline Group: 10 healthy mice are routinely reared for one week; the saline is intraperitoneally injected once daily at a concentration of 10 ml/kg for 7 consecutive days; 15 min after the last injection is finished, 0.7% glacial acetic acid (10 ml/kg) is intraperitoneally injected; and each mouse is observed for their writhing behaviors.

Experimental Group: 10 healthy mice are routinely reared for one week; 10 ml/kg of different polypeptides are injected intraperitoneally at a concentration of 0.1 mL/10 g once daily for 7 consecutive days; 15 min after the last injection is finished, 0.7% glacial acetic acid (10 ml/kg) is intraperitoneally injected; and each mouse is observed for their behaviors in writhing.

During the above experiments, the time of first writhing (writhing latency) and the number of writhing in each mouse within 15 min are recorded immediately after the injection of glacial acetic acid solution in each group of mice. When behavioral responses such as inward concavity of the abdomen, extension of the torso and hind limbs, and elevation of the buttocks occur, it is considered to be a complete writhing response, and the results are shown in Table 3 and FIG. 6.

TABLE 3
Effects of different polypeptides on
the mouse acetate acid writhing model

| | | | | Number of |
|---|---|---|---|---|
| | Polypeptides | Number of | Time of first | writhing |
| Groups | Sequence ID | mice | writhing (min) | (times/15 min) |
| Saline | — | 10 | 2.99 | 31.5 |
| PPN-01 | SEQ ID NO: 1 | 10 | 4.15 | 14.4 |
| PPN-04 | SEQ ID NO: 4 | 10 | 3.64 | 22.1 |
| PPN-06 | SEQ ID NO: 6 | 10 | 3.67 | 16.5 |
| PPN-11 | SEQ ID NO: 11 | 10 | 3.07 | 23.5 |
| PPN-12 | SEQ ID NO: 12 | 10 | 3.15 | 21.7 |
| PPN-16 | SEQ ID NO: 18 | 10 | 2.84 | 22.8 |

As shown in Table 3 and FIG. 6, the number of writhing in the experimental group is less compared with that in the saline group, which indicates that the polypeptides PPN-01, PPN-04, PPN-06, PPN-11, PPN-12, and PPN-16 all provide a certain analgesic effect on the mouse acetate acid writhing model, thereby resulting in a significant reduction in the number of writhing in the mice.

### Example 7 Effects of Different Concentrations of Polypeptide PPN-01 on the Mouse Acetate Acid Writhing Model

The objective of the present example is to study the effect of different concentrations of the polypeptide PPN-01 (the amino acid sequence of which is shown in SEQ ID NO: 3) on the mouse acetate acid writhing model.

Forty SPF-grade healthy C57BL/6 mice, half male and half female, weighing 22-25 g and 8-10 weeks old, are randomly selected. Forty C57BL/6 mice are weighed and numbered in ascending order of body weight (No. 1 to No. 40). The mice are randomly divided into five groups according to the randomized numerical table method, namely, the saline group, the low-concentration group (2.5 mg/kg), the medium-concentration group (10 mg/kg), the high-concentration group (40 mg/kg), and the diclofenac sodium group (30 mg/kg), with eight mice in each group.

Saline Group: 8 healthy mice are routinely reared for one week; the saline is intraperitoneally injected once daily at a concentration of 10 ml/kg for 7 consecutive days; 15 min after the last injection is finished, 0.7% glacial acetic acid (10 ml/kg) is intraperitoneally injected; and each mouse is observed for their behaviors in writhing.

Low-concentration Group (2.5 mg/kg): 8 healthy mice are routinely reared for one week; the polypeptide PPN-01 solution at a concentration of 0.5 μM is intraperitoneally injected once daily at a concentration of 10 ml/kg; the mice are administered with a concentration of 2.5 mg/kg for 7 consecutive days; 15 min after the last injection is finished, 0.7% glacial acetic acid (10 ml/kg) is intraperitoneally injected; and each mouse is observed for their behaviors in writhing.

Medium-concentration Group (10 mg/kg): 8 healthy mice are routinely reared for one week; the polypeptide PPN-01 solution at a concentration of 2 μM is intraperitoneally injected once daily at a concentration of 10 ml/kg; the mice are administered with a concentration of 10 mg/kg for 7 consecutive days; 15 min after the last injection is finished, 0.7% glacial acetic acid (10 ml/kg) is intraperitoneally injected; and each mouse is observed for their behaviors in writhing.

High-concentration Group (40 mg/kg): 8 healthy mice are routinely reared for one week; the polypeptide PPN-01 solution at a concentration of 8 μM is intraperitoneally injected once daily at a concentration of 10 ml/kg; the mice are administered with a concentration of 40 mg/kg for 7 consecutive days; 15 min after the last injection is finished, 0.7% glacial acetic acid (10 ml/kg) is intraperitoneally injected; and each mouse is observed for their behaviors in writhing.

Diclofenac Sodium Group (10 ml/kg): 8 healthy mice are routinely reared for one week; the diclofenac sodium solution at a concentration of 3 mg/ml is intraperitoneally injected once daily at a concentration of 10 ml/kg; the mice are administered with a concentration of 30 mg/kg for 7 consecutive days; 15 min after the last injection is finished, 0.7% glacial acetic acid (10 ml/kg) is intraperitoneally injected; and each mouse is observed for their behaviors in writhing.

During the above experiments, the time of first writhing (writhing latency) and the number of writhing in each mouse within 15 min are recorded immediately after the injection of glacial acetic acid solution in each group of mice. When behavioral responses such as inward concavity of the abdomen, extension of the torso and hind limbs, and elevation of the buttocks occur, it is considered to be a complete writhing response, and the analgesic effects of the polypeptide PPN-01 are assessed by the inhibition rate (%) and analgesic rate (%).

$$Inhibition rate ( \% ) = [ ( mean latency of administered group - mean latency of saline group ) / mean latency of saline group ] \times 100 \%$$

$$Analgesic rate ( \% ) = [ ( mean number of writhing in saline group - mean number of writhing in administered group ) / mean number of writhing in saline group ] \times 100 \%$$

All statistics are performed using one-way ANOVA for statistics between two groups, with P<0.05 as statistically different (* means P<0.05, ** means P<0.01, *** means P<0.001), and all statistics are graphed using Graphpad Prism 9.0 software.

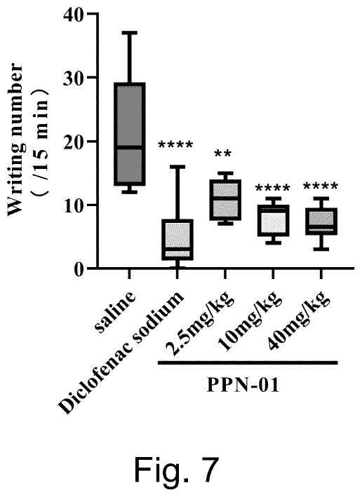

The writhing inhibition rates of different concentrations of polypeptide PPN-01 in each group of mice are shown in Table 4, and the results of the number of writhing in each group of mice after injection of different concentrations of polypeptide PPN-01 are shown in FIG. 7.

TABLE 4
Results of writhing inhibition rate of each group of mice

| Groups | Inhibiting Rate (%) |
|---|---|
| Saline Group | — |
| Diclofenac Sodium Group | 77.6 |
| Low-concentration Group | 43.5 |
| Medium-concentration Group | 72.5 |
| High-concentration Group | 75.7 |

As shown in Table 4 and FIG. 7, after the intervention of diclofenac sodium or different concentrations of the polypeptide PPN-01, the writhing response due to inflammatory pain in mice is decreased, and the inhibition rate of writhing in mice is increased with the increasing concentration of the polypeptide PPN-01 administered, which is up to 75.7% and slightly lower than that in the diclofenac sodium group.

### Example 8 Effects of Polypeptide PPN-01 on Electrical Signals of the Sciatic Nerve in Rats

The objective of the present example is to study the effects of the polypeptide PPN-01 (the amino acid sequence of which is shown in SEQ ID NO: 3) on the electrical signals of the sciatic nerve in rats.

Four SPF-grade healthy Sprague-Dawley (SD) rats are randomly selected, half males and half females, weighing 180˜200 g, 6˜8 weeks old. The rats are first acclimatized and fed for 1 week, fasted for 12 h before the experiment, with free access to water.

Rats weighing between 180 g and 200 g are taken and anesthetized with 7% chloral hydrate solution at a concentration of 0.4 mL/100 g. The anesthesia is complete when the rat does not show the roll-over reflex and no response to tight pinching of the rat's tail. Then, the fully anesthetized rats are placed on the operating table so that their backs are facing upwards and their paws are held in place with a string. The skin and connective tissue of the leg are cut sequentially, and the muscles between the biceps femoris and the gluteus superficialis are bluntly-dissected. The sciatic nerve is further hooked out between the two muscles with a glass needle, and two sutures are passed through each end of the sciatic nerve to facilitate subsequent experiments.

Electrodes specialized for recording nerve impulses are placed on the sciatic nerve, and the electrical signals emitted by nerve impulses are converted into digital signals through a multi-channel physiological signal acquisition and processing system, which are displayed on the computer as nerve compound action potential (CAP). These electrical signals are then acquired by the computer for storage and subsequent analysis. Each experiment is performed alternately between the right and left legs, and the sciatic nerve is moistened with saline from time to time during the experiment to avoid exposing the nerve directly to air for excessive time.

Polypeptide PPN-01 group: The sciatic nerve on one side of the rat is separated and electrodes specialized for recording nerve impulses are placed on that sciatic nerve to record CAP, first stabilized for 5 min, during this time the sciatic nerve required to be moistened with 0.9% saline. Recordings begin after 5 min by aspirating 20 μL of 0.9% saline drops onto the sciatic nerve; recording for 5 min. The soles of the rats' feet are then stimulated with a syringe needle; recording for 5 min again. Then 20 μL of low (30 nM), medium (100 nM), and high (300 nM) concentrations of polypeptide PPN-01 are added sequentially to the sciatic nerves; all the previous reagent is aspirated with a paper towel before the next reagent is added; and the recording is performed in the same way as saline. The sciatic nerve on the other side is first dripped with the polypeptide PPN-01, rinsed and then dripped with saline as a control; the order of dosing and saline is swapped bilaterally in different rats.

Negative control drug lidocaine group: The sciatic nerve on one side of the rat is separated and electrodes specialized for recording nerve impulses are placed on that sciatic nerve to record CAP, first stabilized for 5 min, during this time the sciatic nerve required to be moistened with 0.9% saline. Recordings begin after 5 min by aspirating 20 μL of 0.9% saline drops onto the sciatic nerve; recording for 5 min. The soles of the rats' feet are then stimulated with a syringe needle; recording for 5 min again. 20 μL of lidocaine is dripped on the sciatic nerve; recording for 5 min. Then the soles of the rats' feet are stimulated with a syringe needle; recording for 5 min. Different rats are controlled bilaterally in the same way as above.

Observations are performed on rats in the polypeptide PPN-01 group and in the negative control drug lidocaine group, and the effect of each concentration of polypeptide PPN-01 on the peak amplitude of the positive and negative phases of the CAP in the sciatic nerve of rats is assessed by the inhibition rate. The inhibition rate is calculated in accordance with the following formula: inhibition rate (%)=[(peak amplitude of the dosed group-peak amplitude of the negative control group)/peak amplitude of the negative control group]×100%.

Four to six CAPs within 2 min after each recording time period are selected for analysis, and the mean peak amplitude is calculated as the representative CAP peak amplitude and time course for each group, with the peak amplitude of the CAP being the difference between the baseline and the peak CAP.

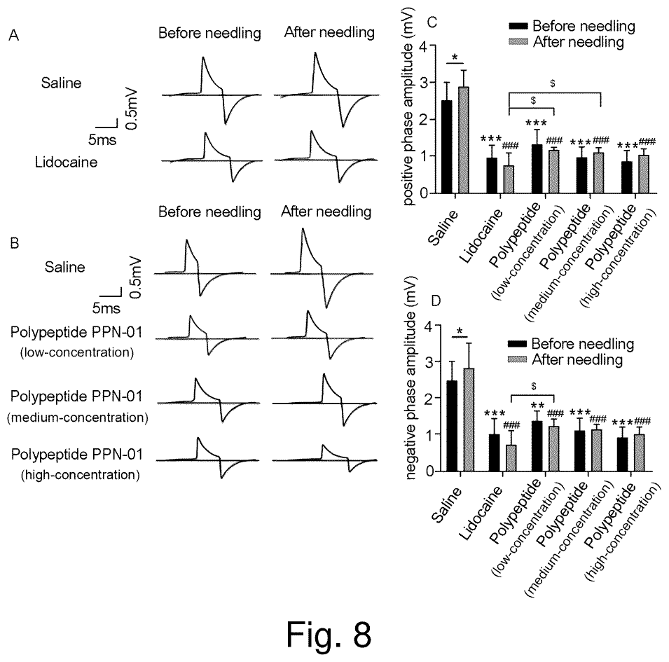

The resulting effects of polypeptide PPN-01 on the electrical signals of rat sciatic nerve are shown in FIG. 8. Panel A of FIG. 8 represents representative traces before and after needling of rat sciatic nerve CAP before and after lidocaine dosing. Panel B of FIG. 8 represents representative traces of rat sciatic nerve CAP before and after needling in groups with different concentrations of polypeptide PPN-01. Panel C of FIG. 8 represents the results of the statistical analysis of the comparison of the positive phase amplitude of CAP before and after needling in rats of different concentrations of polypeptide PPN-01 and lidocaine groups. Panel D of FIG. 8 represents the results of the statistical analysis of the comparison of the negative phase amplitude of CAP before and after needling in rats of different concentrations of polypeptide PPN-01 and lidocaine groups. The above data are presented as mean±standard deviation (x±sd). “*”, “**” and “***” indicate P<0.05, P<0.01, and P<0.001, respectively, compared with the saline group before needling. “###” indicates P<0.001 compared with the saline group after needling. “$” indicates P<0.05 compared with the lidocaine group after needling.

As shown in FIG. 8, the saline group shows the largest peak amplitude before and after needling, and the peak amplitude of each group is decreased to a different extent after the drug intervention. Compared with the negative control group, the peak amplitude of CAP is significantly decreased in all concentrations of PPN-01 groups before and after needling (p<0.05), in which the inhibition of the peak amplitude of the positive and negative phases is greatest in the high-concentration PPN-01 group and least in the low-concentration PPN-01 group. Compared with the negative control drug, lidocaine, the inhibition rate is greatest in the high-concentration PPN-01 group before needling and in the lidocaine group after needling. Compared with the high-concentration PPN-01 group, both the low-concentration PPN-01 group and the medium-concentration PPN-01 group show smaller inhibition rates before and after needling, but the difference between these two groups and the high-concentration PPN-01 group is not statistically significant (p>0.05). The results above indicate that the peak amplitude of positive and negative phases of sciatic nerve CAP in rats is effectively reduced by the polypeptide PPN-01, and such an effect is similar to that of lidocaine (P>0.05). The higher the concentration of the drug, the more significant the effect will be.

In summary, the polypeptide provided in the present disclosure may selectively inhibit an activation of voltage gated Naᵥ1.7 sodium channels, thereby inhibiting pain. By applying the polypeptides provided in the present disclosure to the preparation of analgesic drugs, the produced drugs provide good analgesic effects and are of great clinical importance in pain disorders.

The above examples are only used to illustrate the technical solution of the present disclosure rather than to limit the protection scope of the present disclosure. Although the present disclosure has been described in detail with reference to the above examples, a person of ordinary skill in the art should be aware that modifications or equivalent substitutions may be carried out to the technical solution of the present disclosure, these modifications or substitutions fall within the protection scope of the present disclosure.

What is claimed is:

1. A polypeptide, wherein an amino acid sequence of the polypeptide is as shown in SEQ ID NO: 1 or SEQ ID NO: 2; a 12^{th} amino acid in the amino acid sequence of the polypeptide is alanine or glutamic acid; a 19^{th} amino acid in the amino acid sequence of the polypeptide is methionine or leucine; a 28^{th} amino acid in the amino acid sequence of the polypeptide is lysine or isoleucine; and a 29^{th} amino acid in the amino acid sequence of the polypeptide is selected from any one of isoleucine, leucine and tryptophan.

2. The polypeptide according to claim 1, wherein the amino acid sequence of the polypeptide is selected from any one of SEQ ID NO: 3-18.

3. A nucleic acid molecule, wherein the nucleic acid molecule comprises a nucleotide sequence encoding a polypeptide of claim 1.

4. A nucleic acid molecule, wherein the nucleic acid molecule comprises a nucleotide sequence encoding a polypeptide of claim 2.

5. A recombinant expression vector, wherein the recombinant expression vector comprises a nucleic acid molecule of claim 3.

6. A recombinant expression vector, wherein the recombinant expression vector comprises a nucleic acid molecule of claim 4.

7. A recombinant host cell, wherein the recombinant host cell comprises a recombinant expression vector of claim 5.

8. A recombinant host cell, wherein the recombinant host cell comprises a recombinant expression vector of claim 6.

9. An analgesic drug, comprising a polypeptide of claim 1.

10. An analgesic drug, comprising a polypeptide of claim 2.

11. The analgesic drug according to claim 9, wherein the drug is capable of inhibiting an activation of voltage gated sodium channels.

12. The analgesic drug according to claim 10, wherein the drug is capable of inhibiting an activation of voltage gated sodium channels.

13. The analgesic drug according to claim 11, wherein the voltage gated sodium channels comprise Naᵥ1.7 channels.

14. The analgesic drug according to claim 12, wherein the voltage gated sodium channels comprise Naᵥ1.7 channels.

15. The analgesic drug according to claim 9, wherein a preparation type of the analgesic drug comprises at least one of a colloidal solution type, an emulsion type, a suspension type, an oral instant film, an oral liquid, a capsule, an injection or a transdermal absorption preparation.

16. The analgesic drug according to claim 10, wherein a preparation type of the analgesic drug comprises at least one of a colloidal solution type, an emulsion type, a suspension type, an oral instant film, an oral liquid, a capsule, an injection or a transdermal absorption preparation.

17. A pharmaceutical composition, wherein an active ingredient of the pharmaceutical composition comprises a polypeptide of claim 1.

18. A pharmaceutical composition, wherein an active ingredient of the pharmaceutical composition comprises a polypeptide of claim 2.
